(19) United States
(12) Patent Application Publication
(10) Pub. No.: US 2026/0027103 A1
(43) Pub. Date: Jan. 29, 2026

(54) PHARMACEUTICAL COMPOSITIONS COMPRISING A QUINOLONE COMPOUND FOR IRRITABLE BOWEL SYNDROME

(71) Applicant: OTSUKA PHARMACEUTICAL CO., LTD., Tokyo (JP)

(72) Inventors: Shimei CHEN, Shanghai (CN); Jie WEN, Shanghai (CN); Jay Fei CHENG, Shanghai (CN); Fengzhu LIU, Shanghai (CN); Wei HU, Shanghai (CN)

(73) Assignee: OTSUKA PHARMACEUTICAL CO., LTD., Tokyo (JP)

(21) Appl. No.: 18/993,115

(22) PCT Filed: Jul. 11, 2023
(86) PCT No.: PCT/CN2023/106675

§ 371 Date: Jan. 10, 2025

(30) Foreign Application Priority Data
Jul. 11, 2022 (WO) PCT/CN2022/104866

(51) Int. Cl. A61K 31/4709; A61K 9/00; A61P 1/00

(52) U.S. Cl. CPC A61K 31/4709; A61K 9/0053; A61P 1/00

(57) ABSTRACT

Provided herein are pharmaceutical compositions comprising a quinolone compound for irritable bowel syndrome, especially provided is the use of the quinolone compound, in particular 1-cyclopropyl-6-fluoro-1. 4-dihydro-8-methyl-7-(2-amino-3-cyano-5-pyridyl)-4-oxo-3-quinoline-carboxylic acid or a pharmacologically acceptable salt, a hydrate, a solvate or a deuteride thereof for preventing or treating irritable bowel syndrome of a human or an animal.

### TECHNICAL FIELD

The present invention belongs to a medical field, and in particular pertains to a drug for the prevention or treatment of irritable bowel syndrome, the drug containing a low absorbed quinolone compound or a pharmacologically acceptable salt of the compound as an active ingredient.

### BACKGROUND ART

IBS (Irritable bowel syndrome) is a functional gastrointestinal disorder with symptoms including abdominal pain associated with a change in stool form or frequency, but not associated with structural or biochemical abnormalities that are detectable with the current routine diagnostic tools. The condition affects between 5% and 10% of otherwise healthy individuals at any one point in time, and in most people runs a relapsing and remitting course. According to Roman classification, IBS can be classified into four categories: diarrhea (IBS-D), constipation (IBS-C), mixed (IBS-M) and unknown type (IBS-U). In China, IBS population is in the range of 1.4-11.5%, and majority is diarrhea-dominated (IBS-D). Only 25% of patients visited hospitals seeking a treatment.

There is no currently cure for IBS. The mainstays of treatment include patient education about the condition, dietary changes, soluble fiber, and antispasmodic drugs. Other treatments tend to be reserved for people with severe symptoms and include central neuromodulators, intestinal secretagogues, drugs acting on opioid or 5-HT receptors, or minimally absorbed antibiotics, as well as psychological therapies. For example, tricyclic antidepressant is effective in treating IBS-D, although there are a number of side effects such as sleep, constipation, thirsty etc. Similarly, 5HT antagonists have been adapted in this area (Non Patent Literature 1: Ford et al, Functional Gastrointestinal Disorders. 2020, 396, 1675-1688). Even using these medicaments, however, any complete therapy of IBS-D is not expected, thus it has been desired to develop a more potent medicament thereof than existing medicaments.
Patent Literature 1 (WO2013/029548) discloses specific quinolone antimicrobials which exhibit the antibacterial activity against Clostridioides (Clostridium) difficile living in intestinal tract.

### CONTENTS OF INVENTION

### Technical Problem

The main purpose of the present invention is to provide a novel medicament for treating and/or preventing irritable bowel syndrome (IBS), especially IBS-D, which is more effective than existing medicaments.

### Solution to Problem

The present inventors have extensively studied and then have found that a known quinolone antimicrobial, 1-cyclopropyl-6-fluoro-1,4-dihydro-8-methyl-7-(2-amino-3-cyano-5-pyridyl)-4-oxo-3-quinoline-carboxylic acid can improve defecation volume of restrained rats, which is the hallmark of an irritable bowel syndrome, and is effectable for treatment of an irritable bowel syndrome. Based upon the new findings, the present invention has been completed.

### SUMMARY OF THE INVENTION

The present invention provides use of a quinolone compound for preventing or treating IBS, especially IBS-D, as described in Items 1 to 11 below.
Item 1. A medicament for treating and/or preventing IBS, comprising 1-cyclopropyl-6-fluoro-1,4-dihydro-8-methyl-7-(2-amino-3-cyano-5-pyridyl)-4-oxo-3-quinoline-carboxylic acid or a pharmaceutically acceptable salt, a precursor or metabolite, a hydrate, a solvate or a deuteride thereof as an active ingredient.
Item 2. The medicament of item 1, wherein the metabolite is (2S,3S,4S,5R,6R)-6-((7-amino-5-cyanopyridin-3-yl)-1-cyclopropyl-6-fluoro-8-methyl-4-oxo-1,4-dihydroquinoline-3-carbonyl) oxo)-3,4,5-trihydroxytetrahydro-2H-pyran-2-carboxylic acid, 7-(6-amino-5-carbamoylpyridin-3-yl)-1-cyclopropyl-6-fluoro-8-methyl-4-oxo-1,4-dihydroquinoline-3-carboxylic acid, or ethyl 7-(6-amino-5-cyanopyridin-3-yl)-1-cyclopropyl-6-fluoro-8-methyl-4-oxo-1,4-dihydroquinoline-3-carboxylate.
Item 3. The medicament of item 1 or 2, which is for oral administration.
Item 4. The medicament of any one of items 1-3, wherein a daily dose of the active ingredient is 7.5 mg-24000 mg.
Item 5. The medicament of any one of items 1-4, wherein IBS is IBS-D.
Item 6. A method for treating and/or preventing IBS, comprising administering a therapeutically effective amount of 1-cyclopropyl-6-fluoro-1,4-dihydro-8-methyl-7-(2-amino-3-cyano-5-pyridyl)-4-oxo-3-quinoline-carboxylic acid or a pharmaceutically acceptable salt, a precursor or metabolite, a hydrate, a solvate or a deuteride thereof as an active ingredient to a patient in need thereof.
Item 7. The method of item 6, wherein IBS is IBS-D.
Item 8. Use of 1-cyclopropyl-6-fluoro-1,4-dihydro-8-methyl-7-(2-amino-3-cyano-5-pyridyl)-4-oxo-3-quinoline-carboxylic acid or a pharmaceutically acceptable salt, a precursor or metabolite, a hydrate, a solvate or a deuteride thereof in the manufacture of a medicament for treating and/or preventing IBS.
Item 9. The use of item 8, wherein IBS is IBS-D.
Item 10. 1-Cyclopropyl-6-fluoro-1,4-dihydro-8-methyl-7-(2-amino-3-cyano-5-pyridyl)-4-oxo-3-quinoline-carboxylic acid or a pharmaceutically acceptable salt, a precursor or metabolite, a hydrate, a solvate or a deuteride thereof for use in treating and/or preventing IBS.
Item 11. 1-Cyclopropyl-6-fluoro-1,4-dihydro-8-methyl-7-(2-amino-3-cyano-5-pyridyl)-4-oxo-3-quinoline-carboxylic acid or a pharmaceutically acceptable salt, a precursor or metabolite, a hydrate, a solvate or a deuteride thereof of item 10, wherein IBS is IBS-D.

### DESCRIPTION OF DRAWINGS

FIG. 1 shows an effect of the compound OPS-2071 on body weight of rat in restraint stress RS-IBS model.

FIG. 2 shows an effect of the compound OPS-2071 on body weight change (BWC) % of rat in RS-IBS model.

FIG. 3 shows an effect of the compound OPS-2071 on feces excreted during 2h/day of rat in RS-IBS model.

FIG. 4 shows the visceromotor response in RS-IBS model on day 3, which is one day before compound treatment was recorded between the Control and RS group.

FIG. 5 shows an effect of the compound OPS-2071 on visceromotor response (mmHg) to colorectal distention on day 7 in RS-IBS model.

FIG. 6 shows an effect of the compound OPS-2071 on the histamine level in the urine on day 7 in RS-IBS model.

### DETAILED DESCRIPTION OF THE INVENTION

The compound of the present invention, 1-cyclopropyl-6-fluoro-1,4-dihydro-8-methyl-7-(2-amino-3-cyano-5-pyridyl)-4-oxo-3-quinoline-carboxylic acid having the structure of formula (1), is disclosed as compound 2-18 in WO2013/029548, and can be synthesized according to WO2013/029548.

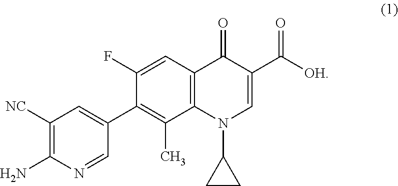 [SMILES: Cc1c(-c2cnc(N)c(C#N)c2)c(F)cc2c(=O)c(C(=O)O)cn(C3CC3)c12]

The compound of the present invention may also include a precursor or metabolite of the compound, e.g., (2S,3S,4S,5R,6R)-6-((7-amino-5-cyanopyridin-3-yl)-1-cyclopropyl-6-fluoro-8-methyl-4-oxo-1,4-dihydroquinoline-3-carbonyl) oxo)-3,4,5-trihydroxytetrahydro-2H-pyran-2-carboxylic acid, 7-(6-amino-5-carbamoylpyridin-3-yl)-1-cyclopropyl-6-fluoro-8-methyl-4-oxo-1,4-dihydroquinoline-3-carboxylic acid, and ethyl 7-(6-amino-5-cyanopyridin-3-yl)-1-cyclopropyl-6-fluoro-8-methyl-4-oxo-1,4-dihydroquinoline-3-carboxylic acid, which have the following structures of formulae (2)-(4), respectively.

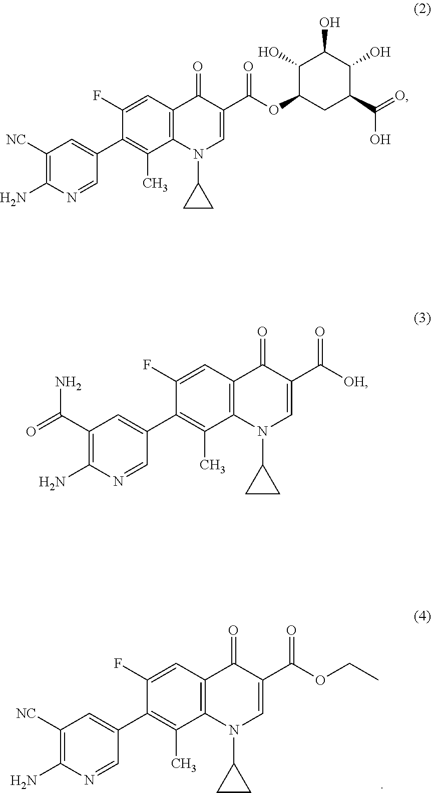 [SMILES: Cc1c(-c2cnc(N)c(C#N)c2)c(F)cc2c(=O)c(C(=O)O[C@@H]3C[C@H](C(=O)O)[C@@H](O)[C@H](O)[C@H]3O)cn(C3CC3)c12 ; Cc1c(-c2cnc(N)c(C(N)=O)c2)c(F)cc2c(=O)c(C(=O)O)cn(C3CC3)c12 ; CCOC(=O)c1cn(C2CC2)c2c(C)c(-c3cnc(N)c(C#N)c3)c(F)cc2c1=O]

The compound of the present invention may be in the form of hydrate and/or solvate, and hence the present compound also encompasses a hydrate and/or solvate thereof. In addition, the compound of the present invention in which any one or more 1H atoms are replaced by 2H (D) atoms is also within the scope of the present invention. There may exist a polymorphism in a crystal of the compound of the present invention or a pharmaceutically acceptable salt thereof, and hence such crystal polymorphism is also within the scope of the present invention.

The “pharmaceutically acceptable salt” includes, as an acid addition salt, a salt with inorganic acid such as hydrochloride, hydrobromide, hydroiodide, sulfate, perchlorate, and phosphate; a salt with organic acid such as oxalate, malonate, maleate, fumarate, lactate, malate, citrate, tartrate, benzoate, trifluoroacetate, acetate, methanesulfonate, ptoluenesulfonate, and trifluoromethanesulfonate; and a salt with amino acid such as glutamate and aspartate; and as a salt with a base, an alkali metal salt such as sodium salt and potassium salt; alkaline-earth metal salt such as calcium salt; and an ammonium salt.

The compound and its pharmaceutical compositions of the present invention are usually used in the form of a usual pharmaceutical preparation. The pharmaceutical preparation can be prepared in admixture with conventional pharmaceutically acceptable diluents or carriers, such as fillers, bulking agents, binding agents, wetting agents, disintegrators, surfactants and lubricating agents. The pharmaceutical preparation includes various preparations suitable for treatment of the diseases, for example, tablets, pills, powders, solutions, suspensions, emulsions, granules, capsules, suppositories, injections such as solutions and suspensions, and the like. In the preparation of tablets, there may be used any conventional carriers, for example, excipients such as lactose, white sugar, sodium chloride, glucose, urea, starches, calcium carbonate, kaolin, crystalline cellulose and silicate, binding agents such as water, ethanol, propanol, simple syrup, glucose solution, starch solution, gelatin solution, carboxymethyl cellulose, shellac, methyl cellulose, potassium phosphate and polyvinylpyrrolidone, disintegrators such as dry starch, sodium alginate, agar powder, laminaran powder, sodium hydrogen carbonate, calcium carbonate, polyoxyethylene sorbitan fatty acid esters, sodium lauryl sulfate, stearic monoglyceride, starches and lactose, disintegration inhibitors such as white sugar, stearin, cacao butter and hydrogenated oils, absorption promoters such as quintenary ammonium salts and sodium lauryl sulfate, wetting agents such as glycerin and starches, adsorbents such as starches, lactose, kaolin, bentonite and colloidal silicates, lubricants such as purified tale, stearates, boric acid powder and polyethylene glycol, and the like. The tablets may also be coated with conventional coating agents, for example, may be in the form of a sugar coated tablet, a gelatin-coated tablets, an enteric coating tablet, a film coating tablet, or a double or multiple layers tablet. In the preparation of pills, there may be used conventional carriers, including excipients such as glucose, lactose, starches, cacao butter, hydrogenated vegetable oils, kaolin and tale, binding agents such as gum arabic powder, tragacanth powder, gelatin and ethanol, disintegrators such as laminaran and agar, and the like. In the preparation of suppositories, there may be used conventional carriers, such as, for example, polyethylene glycol, cacao butter, higher alcohols, higher alcohol esters, gelatin and semi-synthesized glycerides. In the preparation of injections, the solutions, emulsions or suspensions of the compounds are sterilized and are preferably made isotonic with the body liquid. These solutions, emulsions and suspensions are prepared by admixing the active compound with a conventional diluent, such as water, aqueous lactic acid solution, ethyl alcohol, propylene glycol, ethoxylated isostearyl alcohol, polyoxylated isostearyl alcohol or polyoxyethylene sorbitan fatty acid esters. The preparations may also be incorporated with sodium chloride, glucose or glycerin in an amount sufficient to make them isotonic with the body liquid. The preparations may also be incorporated with conventional solubilizers, buffering agents, anesthetizing agents, and further, with coloring agents, preservatives, perfumes, flavors, sweetening agents, and other medicaments. The preparations in the form of a paste, cream or gel may be prepared by using as a diluent such as white petrolatum, paraffin, glycerin, cellulose derivatives, polyethylene glycol, silicone, bentonite, or the like. When the compound of the active ingredient precipitates in the injection, an acid such as, for example, methanesulfonic acid, propionic acid, hydrochloric acid, succinic acid or lactic acid may be added to the injection as required to preserve the injection in a stable solution.

The compounds of the present invention may be contained in any amount in the preparations, and are usually contained in an amount of from 1 to 70% by weight based on the whole weight of the preparations.

The pharmaceutical composition of the present invention can be administered in any methods. Suitable method for administration may be selected in accordance with the preparation form, age and sex of patients, severity of the diseases, and the like. For instance, tablets, pills, solutions, suspensions, emulsions, granules and capsules are administered in oral route In case of injection, it is administered intravenously in a single form or together with an auxiliary liquid such as glucose or amino solution. The injections may also be administered in intramuscular, intracutaneous, subcutaneous, or intraperitoneal route. Suppositories are administered in intrarectal route. The pharmaceutical composition may be in the form of an ophthalmic, an ointment, a patch, and an inhalant

The dosage of the composition of the present invention may vary according to administration methods, age and sex of patients, severity of the diseases, and the like. In case of oral administration, for example, it may be generally administered in a dose of about 0.125 mg-about 400 mg, preferably about 0.25 mg-about 200 mg, more preferably about 0.5 mg-about 100 mg, even more preferably about 1 mg-about 50 mg, per kg of human or mammal's body weight, in one to several portions. For example, the daily dose of human includes about 7.5 mg-about 24000 mg, preferably about 15 mg-about 12000 mg, more preferably about 30 mg-about 6000 mg, even more preferably about 60 mg-about 3000 mg.

The present invention is illustrated by the following Examples, Experimental Examples and Preparation Examples. It is to be understood that the present invention is not limited to these Examples, Experimental Examples of Preparation Examples and various changes and modifications can be made without departing from the scope and spirit of the present invention.

The restrained stress (RS) model used in the Example below is a common model used in examining drug efficiency against IBS, which is first proposed in Williams et al., Gatroenterology 1988, 94:611. The RS model exhibits inhibition of intestinal transport and an increased number of defecation without formation of ulcers, and therefore is considered to mimic IBS which shows a bowel disorder not associated with structural or biochemical abnormalities. Nowadays, improved restraint stress models are widely used, as previously described in Wang et al., Int. J. Gastroenterol. Disord. Ther: 2017, 4:131. References involving visceral hypersensitivity and histamine are, for example, Christine West, Karen-Anne McVey Neufeld, Animal models of visceral pain and the role of the microbiome. Neurobiology of Pain 10 (2021) 100064; Giada De PalmaChiko ShimboriDavid E. ReedYang Yu Virginia RabbiaJun LuNestor Jimenez-VargasJessica SessenweinCintya Lopez-LopezMarc PigrauJosue Jaramillo-Polanco Yong ZhangLauren BaergAhmad Manzar Julien Pujo Xiaopeng BaiMaria Ines Pinto-SanchezAlberto CamineroKaren MadsenMichael G. SuretteMichael BeyakAlan E. LomaxElena F. VerduStephen M. CollinsStephen J. VannerPremysl Bercik. Histamine production by the gut microbiota induces visceral hyperalgesia through histamine 4 receptor signaling in mice, Sci. Transl. Med., 14 (655), eabj 1895.

### SPECIFIC MODE FOR CARRYING OUT THE INVENTION

### Experimental Examples

The present invention is explained in the following in detail by referring to Examples, however, the present invention should not be limited thereto. The present compound used herein (hereinafter, referred to as “test substance”) and the reference drug were obtained as shown below.

### Test Substance

1-cyclopropyl-6-fluoro-1,4-dihydro-8-methyl-7-(2-amino-3-cyano-5-pyridyl)-4-oxo-3-quinoline-carboxylic acid, designated as OPS-2071, was synthesized at Otsuka Pharmaceutical Co., Ltd.

### Experimental Example 1: Effect of Test Substance on Fecal Amount in Restraint Stress (RS) Rat Model

### Animals:

Male Sprague-Dawley (SD) rats, weighting 200-250 g were purchased from Charles River Laboratories Beijing and housed in an SPF grade animal facility of OSRI in individual ventilated cages (IVC), with 3 rats per cage. All animals were maintained with a 12-hour light/dark cycle and had free access to rodent chow and water. All animal study procedures were in accordance with Animal Welfare Regulations and approved by the Institutional Animal Care and Use Committee (IACUC) of OSRI.

### Reagents:

Arabic gum (AG) was purchased from Sigma (CAT #: G9752-500G)

### Establishment of Restraint Stress Rat Model:

SD rats were randomly enrolled into 3 groups based on body weight, with 6 rats per group, as shown in table 1. Rats (from group #2 and group #3) were submitted to a 2-hour restraint in a plastic cylinder daily in the PM for 10 consecutive days (D1 to D10). Rats from the control group (group #1) remained in their individual cages, undisturbed throughout the entire experiment. After 3 days of repeated RS modeling, from D4 to D10, the rats from group #2 and group #3 were treated in the morning (4h ahead of PM restraint) with vehicle (5% AG) and the test substance (2.5 mg/kg) respectively for 7 consecutive days. Fecal pellet output was used to estimate distal colonic motility as a validated index (Hong S et al., Gut 2009, 58:202-210). During the 2-hour restraint period, the rat's fecal pellet output was collected and counted, same for the rats in control group during the same 2-hour mock observation period.

TABLE 1
Description of control group and treatment group of RS model in Example 1

| | | Vehicle or | | | |
|---|---|---|---|---|---|
| | | treatment | Dose | Dosing | Administration |
| Group | Description | (n/group) | (mg/kg) | frequency | route |
| 1 | Non-RS Control group | 5% AG (6) | — | QD*7 (D 4-D 7) | PO |
| 2 | RS model Vehicle group (D 1-D 7) | 5% AG (6) | — | QD*7 (D 4-D 7) | PO |
| 3 | RS model test group (D 1-D 7) | OPS-2071 (6) | 2.5 | QD*7 (D 4-D 7) | PO |

### Results

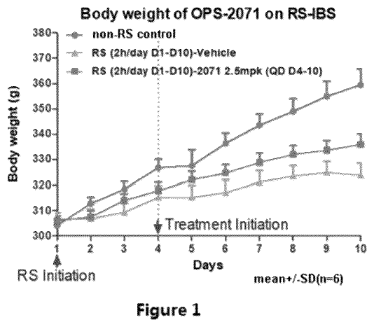

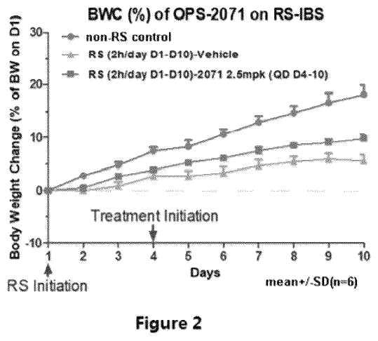

The rat behavior was observed daily and all indexes were normal. As shown in FIGS. 1 and 2, the body weight drop caused by restraint stress is improved by the treatment of OPS-2071. RS modeling at 2h per day for 7 days slowed down the body weight increased observed in non-RS control rats (group #2 vs. group #1). However, OPS-2071 treatment at 2.5 mg/kg for 7 days helped to relieve that effect to certain degree (group #3 vs. group #2).

Food intake was monitored from 3 days post treatment initiation (6 days post RS application). OPS-2071 treatment shown protective effect on decreased appetite observed in RS control group (group #3 vs. group #2).

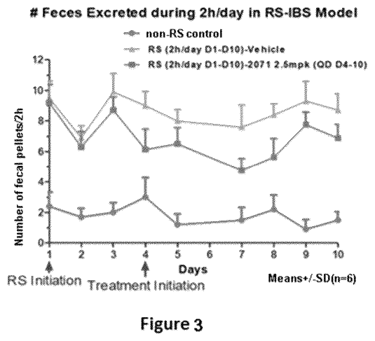

Change of defecation amount were observed upon the placement of restraint stress. Stress caused the increase of fecal pellet output in the RS group (group #2 vs. group #1). At day 4, test group was treated with OPS-2071 at 2.5 mg/kg and a decrease of the fecal pellet output was observed (group #3 vs. group #2), as shown in FIG. 3.

### Experimental Example 2: Evaluation of OPS-2071 Efficacy in Visceral Sensitivity & Histamine Level in Restraint Stress (RS) Model

### Establishment of Restraint Stress Rat RS Model

Sprague Dawley Rats were submitted to a 2h restraint in a plastic cylinder daily for 7 consecutive days as described above. In control group, the rats were remained in an individual cage, undisturbed, throughout the experiment.

### Visceromotor Response to Colorectal Distention on Days 3 and 7

Colonic distension—A polyethylene balloon (3 cm long, 1.5 cm max. diameter) was secured to tubing attached to a blood pressure meter to be used to perform colonic distension. The balloon was lubricated and placed into the rat's distal colon so that the tip of the balloon was 2 cm proximal to the anus. Using the blood pressure meter distension device, rats were restrained in a plastic containment device and allowed to acclimate for 10 min before testing. The rats received phasic distension (0-100 mmHg in 5-mmHg ascending increments) of the colon until the first contraction of the testicles, tail or abdominal musculature occurred. This visualization was performed by three independent observers. The visceral pain threshold indicative was defined as the first nociceptive response as previously described (T. J. Ness; G. F. Gebhart, “Colorectal distension as a noxious visceral stimulus: physiologic and pharmacologic characterization of pseudaffective reflexes in the rat”, Brain Research, Vol. 450, Issues 1-2, pages 153-169, May 21, 1988; Wesselmann, U.; Lai, J., “Mechanisums of referred viiceral pain: uterine inflammation in the adult virgin rat results in neurogenic plasma extravasation in the skin”, Pain Vol. 73, Issue 3, pages 309-317, 1997). Compliance of the colon was not measured given the rapid distension protocol used in this study. The colonic distensions were repeated three times with 5-min interstimulus intervals and the mean pressures at the nociceptive threshold were recorded for each rat.

### Treatment

OPS-2071 (2071): prepared in 5% AG (Arabic gum) once a week, Gum arabic from acacia tree purchased from SIGMA (CAT #: G9752-500G), treatment was initiated from Day 4 to Day 7, as shown in table 2.

TABLE 2
Description of control group and treatment group of RS model in Example 2

| | | | Vehicle or | Dose | Dosing | Administration |
|---|---|---|---|---|---|---|
| Group | Description | # Animals | Treatment | (mg/kg) | frequency | route |
| 1 | Control | 8 | 5% AG | — | QD*7 days | PO |
| 2 | RS-Vehicle | 8 | 5% AG | — | QD*7 days | PO |
| 3 | RS | 8 | OPS-2071 | 2.5 | QD*4 days | PO |

### Sampling

### Urine Collection on Day 7:

Urine samples were collected every day after the restraint. A plastic dish was placed underneath restraint tube and the urine sample was collected. It is immediately transferred into a tube and stored at-800C until further analysis. Histamine was measured with the Histamine ELISA Kit ((CAT #: D751012, BBI) according to the manufacturer's instructions as follows.

### Procedures:

1. Calculate the required number of slats in advance, and take out the kit 30 minutes before the test and restore it to room temperature.
2. Add 50 μL of standard working liquid and test sample to each reaction hole. The standard needs to be re-drilled. 50 μL of biotin-labeled histamine antibody working solution was added to each reaction well immediately, and the plate was sealed and incubated at 37° C. for 45 min.
3. Washing: discard the liquid and shake it dry. Add 350 μL washing liquid into each reaction hole, soak for 1-2 min, shake it dry and repeat 4 times.
4. Add 100 μL of HRP labeled Streptavidin working solution to each reaction well, seal the plate and incubate at 37° C. for 30 min.
5. Washing: Add 300 μL washing liquid into each reaction hole, and dry the liquid at an interval of 30s. Repeat 4 times.
6. Add 90 μL color developing agent (light repellent) into each reaction hole, and develop color repellent at 37° C. for 15 min after plate sealing.
7. Add 50 μL of termination solution into each reaction well, and immediately measure OD value with 450 nm wavelength of enzyme label instrument (within 5 min).
8. OD value was measured with 450 nm wavelength of enzyme label instrument.
9. Draw a standard curve with the concentration of standard substance as the horizontal coordinate and absorbance as the vertical coordinate.
10. Statistical significance was either calculated by two-tailed T-test (visceral sensitivity on day 3) or one-way ANOVA using Dunnett's multiple comparison. P<0.05 was considered statistically significant.

### Results:

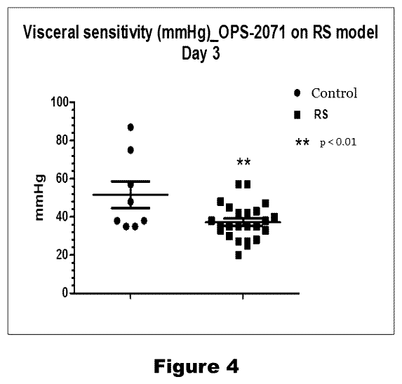

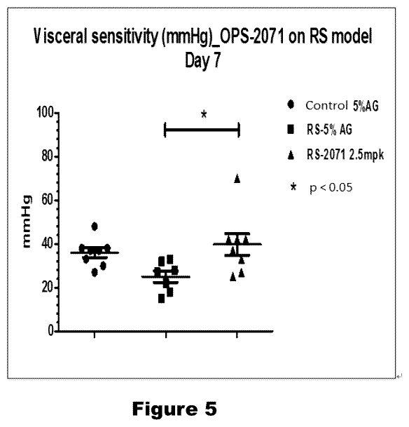

FIGS. 4 and 5 show visceromotor response (mmHg) to colorectal distention, wherein FIG. 4 shows the visceromotor response on day 3, which is one day before compound treatment was recorded between the Control and RS group; FIG. 5 shows the visceromotor response on day 7.

On day 3, the visceral sensitivity of RS group rats increased compared to the Control group, as shown in FIG. 4 (the visceral sensitivity level showed significant difference (p<0.01)—Control group vs RS group).

The treatment group receiving 2.5 mg/kg of OPS-2071 observed a decrease in visceral sensitivity on day 7, as shown in FIG. 5 (i.e. 4 days post dose with OPS-2071; the visceral sensitivity level showed significant difference (p<0.05)—the treated group (2.5 mg/kg) vs RS group).

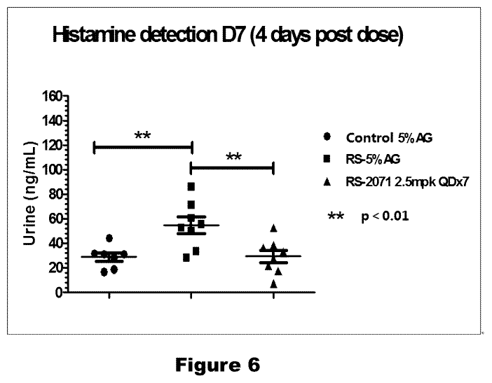

FIG. 6 shows the histamine level in the urine quantified by ELISA on day 7, which is 4 days post OPS-2071 compound treatment.

On day 7, the urine histamine level of RS group rats was higher than that of Control group, while the urine histamine levels observed in the 2.5 mg/kg compound treatment group decreased, as shown in FIG. 6 (the urine histamine level showed highly significant difference (p<0.01) between the control group and RS group; and the urine histamine level showed highly significant difference (p<0.01) between the treated group (2.5 mg/kg) and RS group).

1. A medicament for treating and/or preventing IBS, comprising 1-cyclopropyl-6-fluoro-1,4-dihydro-8-methyl-7-(2-amino-3-cyano-5-pyridyl)-4-oxo-3-quinoline-carboxylic acid or a pharmaceutically acceptable salt, a precursor or metabolite, a hydrate, a solvate or a deuteride thereof as an active ingredient.

2. The medicament of claim 1, wherein the metabolite is (2S,3S,4S,5R,6R)-6-((7-amino-5-cyanopyridin-3-yl)-1-cyclopropyl-6-fluoro-8-methyl-4-oxo-1,4-dihydroquinoline-3-carbonyl) oxo)-3,4,5-trihydroxytetrahydro-2H-pyran-2-carboxylic acid, 7-(6-amino-5-carbamoylpyridin-3-yl)-1-cyclopropyl-6-fluoro-8-methyl-4-oxo-1,4-dihydroquinoline-3-carboxylic acid, or ethyl 7-(6-amino-5-cyanopyridin-3-yl)-1-cyclopropyl-6-fluoro-8-methyl-4-oxo-1,4-dihydroquinoline-3-carboxylate.

3. The medicament of claim 1, which is for oral administration.

4. The medicament of claim 1, wherein the daily dose of the active ingredient is 7.5 mg-24000 mg.

5. The medicament of claim 1, wherein IBS is IBS-D.

6. A method for treating and/or preventing IBS, comprising administering a therapeutically effective amount of 1-cyclopropyl-6-fluoro-1,4-dihydro-8-methyl-7-(2-amino-3-cyano-5-pyridyl)-4-oxo-3-quinoline-carboxylic acid or a pharmaceutically acceptable salt, a precursor or metabolite, a hydrate, a solvate or a deuteride thereof as an active ingredient to a patient in need thereof.

7. The method of claim 6, wherein IBS is IBS-D.

8. Use of 1-cyclopropyl-6-fluoro-1,4-dihydro-8-methyl-7-(2-amino-3-cyano-5-pyridyl)-4-oxo-3-quinoline-carboxylic acid or a pharmaceutically acceptable salt, a precursor or metabolite, a hydrate, a solvate or a deuteride thereof in the manufacture of a medicament for treating and/or preventing IBS.

9. The use of claim 8, wherein IBS is IBS-D.

10. 1-Cyclopropyl-6-fluoro-1,4-dihydro-8-methyl-7-(2-amino-3-cyano-5-pyridyl)-4-oxo-3-quinoline-carboxylic acid or a pharmaceutically acceptable salt, a precursor or metabolite, a hydrate, a solvate or a deuteride thereof for use in treating and/or preventing IBS.

11. 1-Cyclopropyl-6-fluoro-1,4-dihydro-8-methyl-7-(2-amino-3-cyano-5-pyridyl)-4-oxo-3-quinoline-carboxylic acid or a pharmaceutically acceptable salt, a precursor or metabolite, a hydrate, a solvate or a deuteride thereof of claim 10, wherein IBS is IBS-D.
